(19) United States
(12) Patent Application Publication
(10) Pub. No.: US 2002/0082223 A1
(43) Pub. Date: Jun. 27, 2002

(54) Methods of treating cancer using flavonoid glycosides and steroidal glycosides

(76) Inventors: Kejian Fu, Brampton (CA); Jessica Liu, Brampton (CA); Dong Xie, Rockville, MD (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 09/840,537

(22) Filed: Apr. 23, 2001

Related U.S. Application Data
(63) Continuation-in-part of application No. 09/578,649, filed on May 25, 2000.

(51) Int. Cl. A61K 31/7048; A61K 31/704

(52) U.S. Cl. 514/26; 514/27

(57) ABSTRACT

The invention relates to the use of flavonoid glycosides and steroidal glycosides from Hosta or their derivatives as functional ingredients in functional food, OTC and pharmaceutical composition to prevent or treat cancer. The invention further includes methods for making and methods for using the invention.

This application is a continuation-in-part of application Ser. No. 09/578,649, filed May 25, 2000, the entire disclosure of which is incorporated by reference herein.

### FIELD OF THE INVENTION

The invention relates to the use of novel flavonoid and steroidal glycosidic compounds from HOSTA for preventing and treating cancer. The invention further includes methods for making and methods for using the invention.

### BACKGROUND OF THE INVENTION

Since ancient times a vast number of natural remedies of plant and animal origin has been used for medical treatment and disease prevention. The famous Chinese medicine Encyclopaedia “Ben Cao Gang Mu” (52 Volumes) was written by Li Shih Zhen and published in 1596 [1]. This book described 1892 drugs shape, character, taste, source, method of collection and preparation and treatment, prevention of various disease.

A fast growing body of evidence obtained in the recent years by utilization of modem scientific, experimental and clinical methods confirms the biological activity of many micro components of plants that can be utilized in prevention or treatment of a variety of chronic diseases, including cancer and cardiovascular disease [2-4].

Despite the fact that scientific evaluation of medicinal plants historically has been responsible for discovery of a multitude of modern medicine, approximately only 1% of plants has been analyzed so far.

The analysis of botanical material is not a trivial matter. Usually, a sample to be analyzed contains a very complex mixture of many components. Only some of them might be biologically active, while other may be toxic. Components of these complex mixtures are usually interacting amongst themselves . Frequently, in many plants, dozens of-species and strains of the same genus, differ substantially in content of the active ingredients. Even within the same plant, different parts often have different chemical composition. Furthermore, the presence and concentration of some substances depend greatly on the soil, location, season, time of harvest, storage conditions, handling methods, conditions and solvents used for extraction, etc. This diversity of important conditions affecting the quality of botanical remedies requires therefore implementation of stringent, well-designed and closely-monitored standard operating procedures of manufacturing to ensure consistency from batch to batch of a nutraceutical product, followed by application of an appropriate analysis to ensure consistent potency and efficacy.

The major aims of qualitative analyses in phytochemistry include monitoring of the preparative isolation and purification of phytochemicals, chemotaxonomic testing and drug identification and/or detection of adulterants [5-6].

Plant constituents often exist in the form of glycosides. These conjugates may or may not occur together with their respective aglycones. Many glycosides play an important role as drugs and dyes. Glycosides are thermally labile, polar and non-volatile compounds frequently differing in their solubility and biological activity from their respective aglycones. By changing a type and/or number of attached saccharides the physicochemical and biological properties of the glycosides can be modified [7].

Among phytochemicals existing in the glycosilated form that deserve a special attention due to their wide distribution in nature and a high number of beneficial biological and medicinal properties, are saponins and flavonoid glycosides. Saponins are high molecular weight glycosides consisting of a sugar moiety linked to triterpine or steroid aglycones [8]. The most common sugars encountered in saponins are hexoses (glucose, galactose and mannose), 6-deoxyhexoses (rhamnose), pentose (arabinose and xylose), uronic acids (glucuronic acid and galacturonic acid) or amino sugars (glucosamine and galactosamine). Sugars may be linked to the sapogenin at one or two glycosylation sites (through an ether or/and an ester linkage), giving the corresponding monodesmodic or bidesmosidic saponins, respectively [9-10].

Because of the glycosylation of their hydrophobic aglycones, saponins act as biological detergents and, when agitated with water, form a soapy lather that gives rise to name of this group of compound [8]. From a biological point of view saponins have diverse group properties, some deleterious, but many beneficial. Some saponins have been used as plant drugs in folk medicine. They may exhibit cardiac activity, hemolytic activity, activity as fish poisons, hypocholesterolemic [11] immunostimulatory and anti-tumorigenic activity [8]. They can be used as bitterness and sweetness modifiers, allelochemicals and cosmetic ingredients. The second important classes of phytochemicals, which attracted a high interest due to its wide distribution in nature and diversified biological properties are flavonoids [12-13]. These polyphenolic compounds, apart from catechins and proanthocyanidins, consist mainly of glycosides of flavonols, flavons, flavanones, anthocyanins and less frequently isoflavons or free aglycones. Flavonoids represent an important constituent of many edible plants and are present in foods and beverages derived from plants.

Some flavonoid-containing species have been used in traditional medicine. Recently these phytomedicines have been extensively investigated and their health benefits confirmed in many cases for the long-term treatment of mild and chronic diseases or in attaining and maintaining a condition of well-being. Flavonoids function as strong antioxidants [3], free-radical scavengers, and metal chelators and their biological properties can also be linked with their interaction with enzymes, adenosine receptors, and biomembranes [14-15]. Many of the bioflavonoids exhibit very beneficial pharmacological activities, such as anti-inflammatory, antiallergic, antimicrobial, antioxidative, enzyme-inhibitory effects, and etc. [3]

The identification of individual flavonoids, sapogenins and their glycosides has long been carried out by Mass Spectrometry [8, 12, 16], Ultraviolet Spectroscopy [8, 12, 17] and 13C-NMR [8, 18]. These techniques were executed on highly purified compounds and were not applied to mixtures.

Separation of individual flavonoids, sapogenins and their glycosides from each other has long been carried out by Paper, Thin Layer and Open Column Chromatography [8, 12].

More recently HPLC has been used for the separation of individual flavonoids, sapogenins and their glycosides from each other [8, 12 ]. These techniques gave limited resolution between individual glycosides of either the flavonoids or the sapogenins.

The combination of HPLC and Diode Array UV-Visible Detection gave new possibilities in qualitative analysis of flavonoids in plant extracts [19-20]. Information about the type and number of glycosidic units was lost due to the preparation. The extraction and purification did not address the identification and quantization of individual glycosides. Mass Spectrometry with thermospray Ionization permitted routine online analysis of a number of glycosides of both flavonoid and sapogenin classes, but sensitivity was limited and interpretation was complicated by the frequent formation of artifacts [21-22]. Moreover, due to the relatively energetic ionization of the thermospray technique the higher glycosides were not observed. Continuous Flow Fast Atom Bombardment gave some advantages in ionization of small polar molecules but at the cost of instrumental complexity and reliability [23].

The advent of Electrospray Ionization permitted molecules to be ionized with very low energies under atmospheric pressures and at room temperatures. Very polar, high molecular weight species could be routinely analyzed with little artifact formation that could complicate interpretation [24-25]. The technique also permits the use of Collision Induced Fragmentation for generating ions that aids in structure elucidation [26].

The combination of three powerful techniques LC/DAD/ESIMS was used to study the aglycones and glycosides present in berries [24]. These works however largely concentrated on the identification of the flavonoid aglycones or of glycosides of no greater than two units.

The present invention provides a fast and reliable method for the simultaneous analysis of both flavonoid glycosides and steroidal glycosides in one procedure. As a model to show the usefulness of this technique we have chosen plants from Hosta genus which belongs to the subfamily Asphodeloideae in Liliaceae. These plants are widely distributed thus offering easy and economical access to this source of flavonoid and steroidal glycosides of potential medicinal application. The flowers, leaves and rhizomata of hosta have been used as a folk medicine in China [1, 27]. A steroidal saponin identified as hexasaccharide and prepared from the extract of dried Hosta leaves by O. Masamitsu, et al. [28-29] exhibit antibacterial and antitumor activity while some of the steroidal glycosides identified by M. Mimaki group displayed cytostatic activity on HL-60 cells.

Although eight kaempferol glycosides [30] and twenty six steroidal glycosides [28-29, 31-37] have been previously separated from Hosta leaves and Hosta rhizomers, respectively, there has been no reports of any comprehensive procedure to extract simultaneously both classes of glycosides from the Hosta leaves.

### SUMMARY OF THE INVENTION

The present invention is based on the discovery of novel flavonoid glycosides and steroidal glycosides from Hosta that exhibit anti-cancer activities. The present invention provides a method to prevent or treat susceptible cancers in humans comprising administering a cancer-treating amount of these compounds. Preferably, the present method will be utilized to treat or prevent chronic myelogenous leukemia, liver cancer and lung cancer. Another aspect of the present invention is a method as disclosed which utilizes oral or intravenous administration.

### BRIEF DESCRIPTION OF THE DRAWINGS

FIG. 1 is a graph illustrating details of the instrumental setup.

FIG. 2 is a graph showing an application of Collisionally Induced Dissociation for the identification of sugars from a steroidal hexaglycoside where the steroidal aglycone corresponds to an open spiro ring sapogenin and the losses of m/e 162 fragment indicates the presence of five hexose (most likely glucose) moieties and the loss of m/e 146 fragment indicates the presence of one 6- deoxyhexose (most likely α-L-rhamnose).

FIG. 3 is a graph showing an application of Collisionally Induced Dissociation for the identification of sugars from a flavonoid tetraglycoside where the flavonoid aglycone corresponds to kaempferol and the loss of m/e 162 fragment indicates the presence of hexose (most likely glucose).

FIG. 4 is a graph depicting chemical structures of eight kaempferol glycosides, previously found in the extract of Hosta leaves.

FIG. 5 is a graph depicting chemical structures of four steroidal glycosides, previously found in the extract of Hosta rhizomers.

FIG. 6 is a table containing a list of all flavonoid and steroidal glycosides from the Hosta leaves extracts found by the application of this invention, and compared with glycosides previously identified in the literature.

FIG. 7 is a table containing a list of eight new glycosidic compounds identified for the first time in the Hosta leaves extracts by application of the presented procedure.

FIG. 8 is a graph that summarizes the procedure used for extraction, pre-purification and analyses of Hosta leaves in Example 1.

FIG. 9 is a LC/MS chromatogram of the raw extract from Example 1.

FIG. 10 is a graph that summarizes the procedure used for extraction, prepurification and analyses of Hosta leaves in Example 2.

FIG. 11 is a LC/MS chromatogram of a pre-purified mixture of flavonoid and steroidal glycosides extract from Example 2.

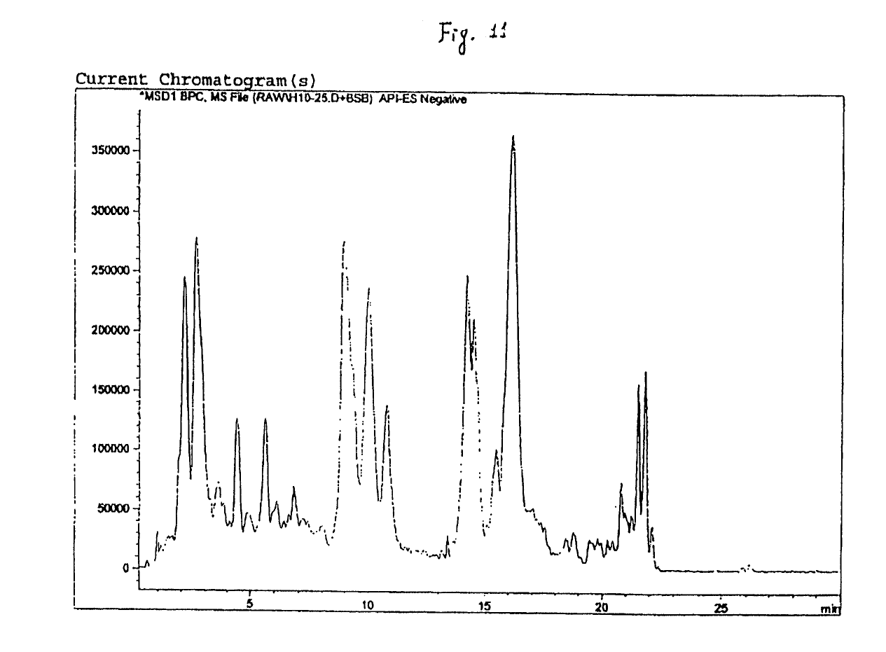

FIG. 12 is a graph which summarizes the procedure used for extraction, prepurification and analyses of Hosta leaves in Example 3

FIG. 13 is a LC/MS chromatogram of the raw extract from Example 3.

FIG. 14 is the result of a cell assay that showed specific anti-cancer activity of S1, an extract containing primarily a steroidal glycosides of molecular weight 1,406 Da. This sample exhibits considerable anti-proliferation activity against three cell lines from CML, liver, and lung cancer.

FIG. 15 is the result of a cell assay that showed specific anti-cancer activity of F2-2, a flavonoid glycoside mixture exhibited selective activity on inhibition of a lung cancer cell line

### DETAILED DESCRIPTION OF THE PREFERRED EMBODIMENT

The present invention is based on the discovery of novel flavonoid glycosides and steroidal glycosides from Hosta that exhibit anti-cancer activities. The present invention is applicable, but not limited, to the extraction, isolation and simultaneous determination of both flavonoid and steroidal glycosides from plants, fresh or dried, and other potential natural sources of nutraceuticals. This procedure is also fully applicable to qualitative and quantitative analysis of different forms of herbal supplementations, such as powders, tinctures, suspensions, solutions, syrups, capsules, tablets, etc.

The biological material, such as plant, should be collected and stored under properly designed and controlled conditions, to ensure the consistency in the content of active components. Measures should be applied to avoid the presence of any harmful contaminants, such as pesticides, herbicides, heavy metals, etc. If drying is recommended, the chemical and enzymatic sensitivity of the active components should be considered. Thus the avoidance of exposure to light, elevated temperatures, oxygen presence or prolonged storage in aqueous solution prone to facilitate biochemical degradation should be considered and applied, if necessary.

Prior to the extraction procedure sample should be prepared by chopping into small pieces, blending, grounding or crushing, in order to improve the contact of solvent with the extracted matter.

In the present invention, for analytical purposes, the extraction of the polar phytochemicals such as bioflavonoids or saponins from the plant or marine matter or their formulations is achieved by the application of water or aqueous solutions of a variety of polar solvents such as lower alcohols, ketones, or acetonitrile. The elevated temperature and repeated extraction procedures might be used to improve the extraction effectiveness. The effectiveness can be further enhanced by stirring, shaking or sonication.

Plant or marine matter consist of a multitude of components, both polar and nonpolar. In order to provide a simple, sensitive and reliable method for analyzing for phytochemicals, such as flavonoid and steroidal glycosides, the sample should be treated to remove contaminants and other undesired components that would interfere with the analysis. Such a removal, for analytical purposes, can be achieved by precipitation of some undesired compounds by means of concentration of the extract volume and/or refrigeration.

Further removal of interfering less polar compounds can be achieved by subjecting the crude extract to liquid-liquid extraction with a water immiscible organic solvent, such as alkanes, cycloalkanes, ethers or lower esters.

The purification of sample from polar components such as inorganic salts, simple sugars, and aminoacids that could interfere with the final analysis of the phytochemicals of interest can be achieved, for analytical purposes, by application of open column or flash column chromatography using a variety of different stationary phases, such as polyamide resin or a weakly acidic cation exchange resin, such as Amberlite IRC-50, and mixture of water and lower alcohols as mobile phase.

Such separation can be easily monitored by HPLC or TLC with a variety of detection methods. The column chromatography may also utilize other modes, such as normal phase chromatography with application of silica gel or alumina or gel filtration approach.

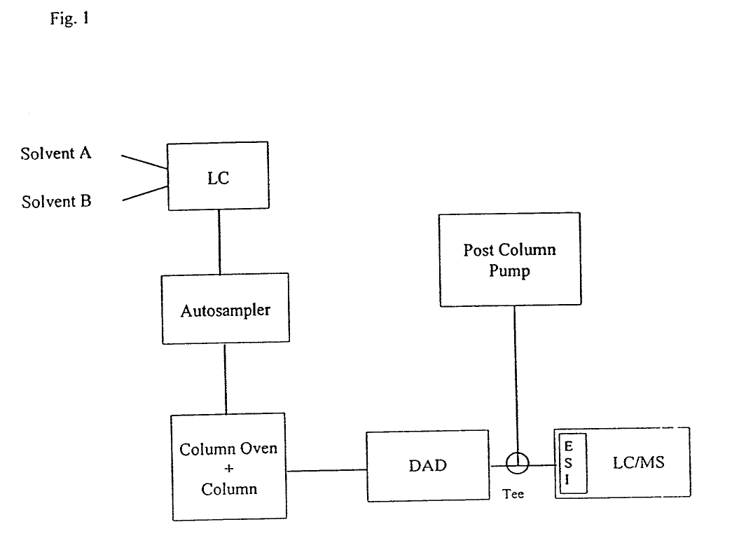

Thus such extracted and pre-purified sample can be then subjected to qualitative and quantitative analysis by the application of reverse phase HPLC in an isocratic or preferably gradient mode. The combined application of two independent and powerful detection techniques of Electrospray Mass Spectrometry and Diode Array Spectroscopy allow for the selective and simultaneous identification of the individual components, such as phytochemicals of interest, contained in the pre-purified extract. The application of Electrospray Mass Spectrometry detection for the thermally liable compounds prevents creation of artifacts that may lead to misinterpretation of the instrumental data that was frequently possible with the previously used ionization techniques. It was found that the application of negative ion mode yields patterns that are more informative than other techniques, decreasing at the same time the risk of the artifact formation and data misinterpretation. Furthermore, the post-column application of triethylamine enhances the sensitivity of this method of detection. FIG. 1 details the instrumental setup.

The sample preparation combined with the applied detection system presented in this invention yield sensitive and extensive qualitative information about the individual components of the analyzed extract, such as, for example flavonoid and steroidal glycosides. This information includes, but is not limited, to molecular weight, number and type of glycoside substituents, and from the Diode Array Spectroscopy absorption patterns, this technique allows for differentiation amongst different types of aglycones present in the components of the extract.

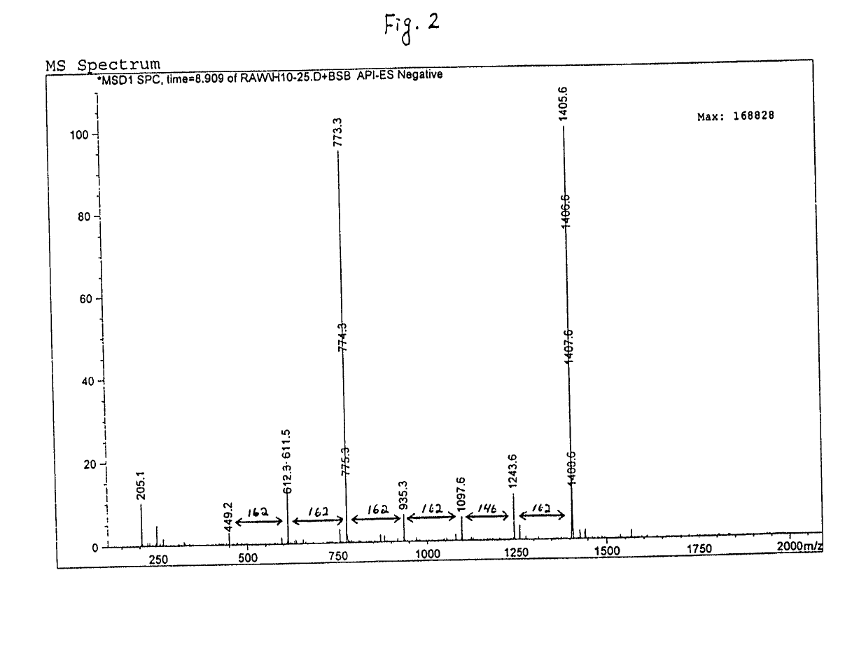

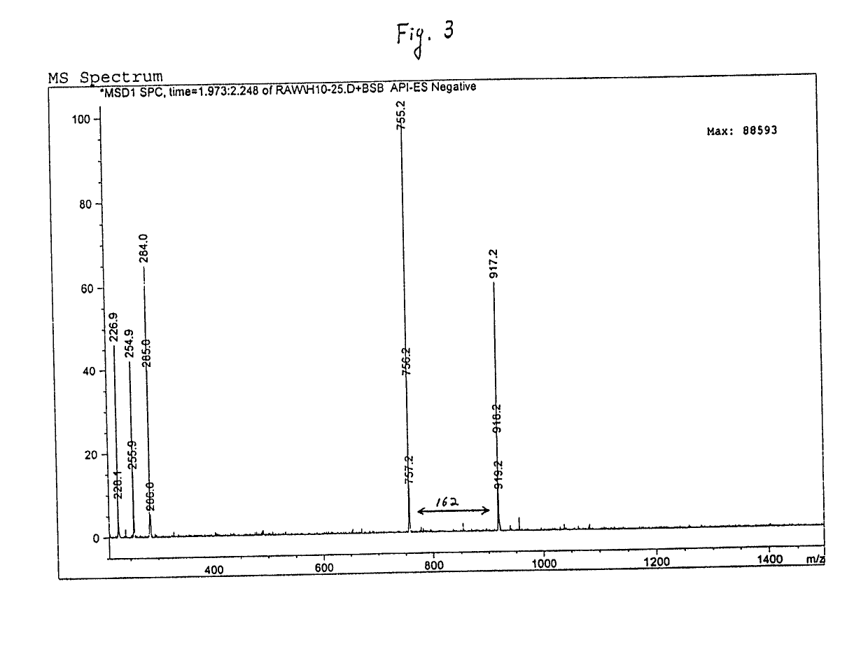

The application of the presented procedure in combination with the use of Collisionally Induced Dissociation can produce mass spectral patterns that can allow structural data to be deduced. Those skilled in the art will recognize that Mile 162 fragment could be related to a loss of hexose (e.g. glucose), that of m/e 146 fragment from the loss of 6-deoxyhexoses (e.g. rhamnose), and that of m/e 132 fragment from the loss of pentose (e.g. xylose) (see FIG. 2 and FIG. 3).

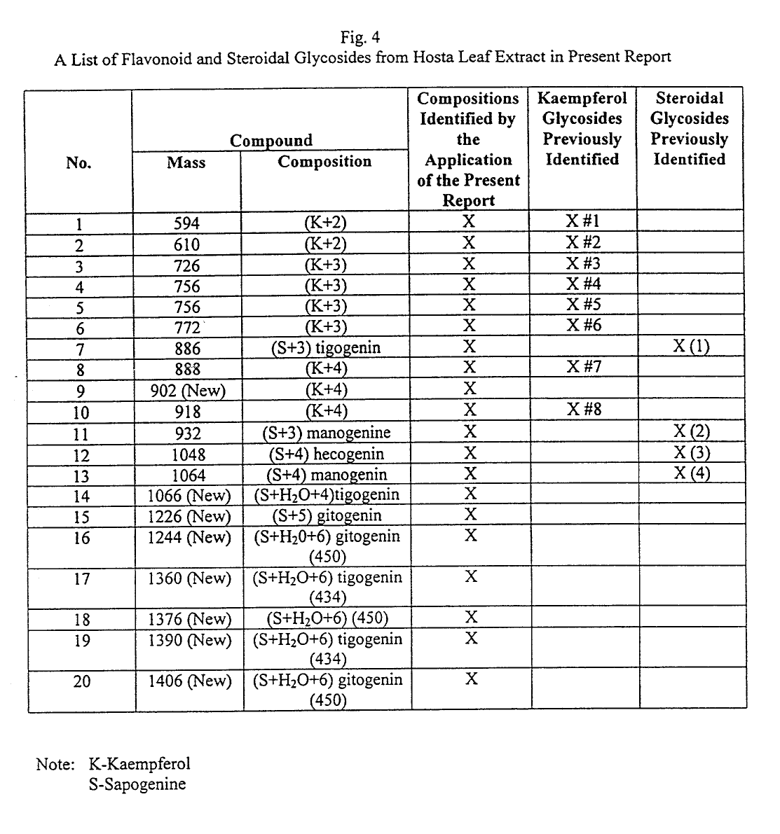

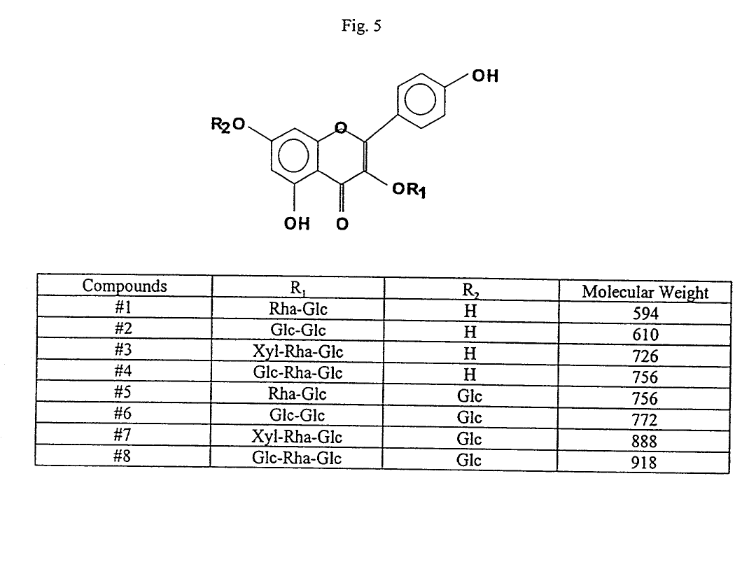

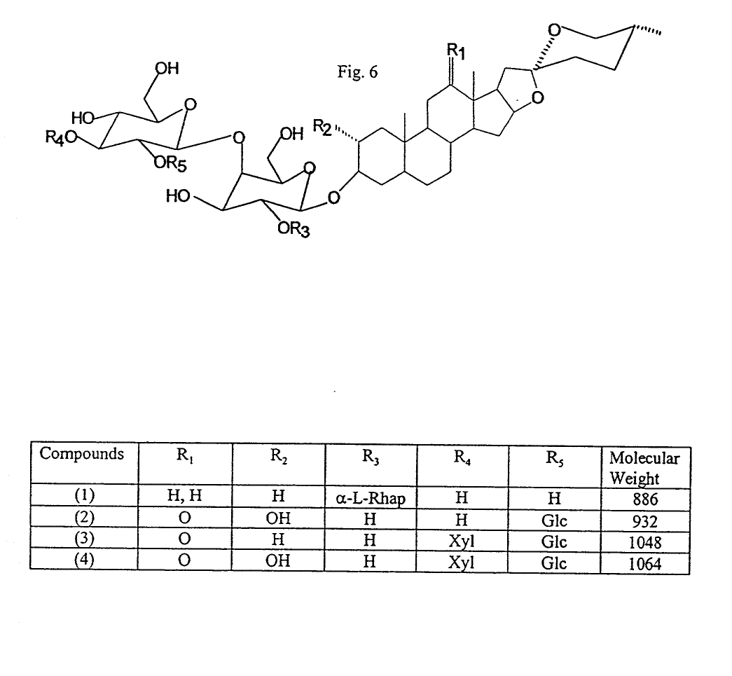

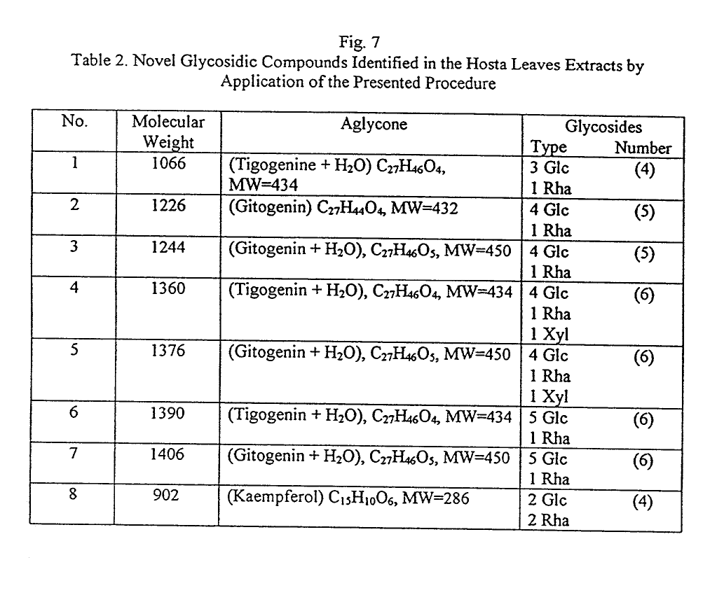

As a very educational and powerful example for the application of this invention can serve the application of the present procedure for the extraction, purification and analysis of flavonoid and steroidal glycosides from Hosta leaves. Thus this invention has allowed for the first time simultaneously identify a total of twenty glycosides, both from the flavonoid and steroidal class (Table 1, FIG. 4). Among the identified glycosidic compounds were all eight kaempferol glycosides, previously reported by J. Budzianowski (structures of which are shown in FIG. 5), and 4 steroidal glycosides, previously reported in the literature by the Japanese researchers for the extracts of Hosta rhizomers (structures of which are shown in FIG. 6). This procedure, with application of Collisionaly Induced Dissociation also allowed for the identification of eight new, previously not detected in the Hosta plant extracts clycosidic compounds, seven of which were identified as steroidal tetra, penta, and hexaglycosides, and one as kaempferol tetraglycoside (Table 2, FIG. 7).

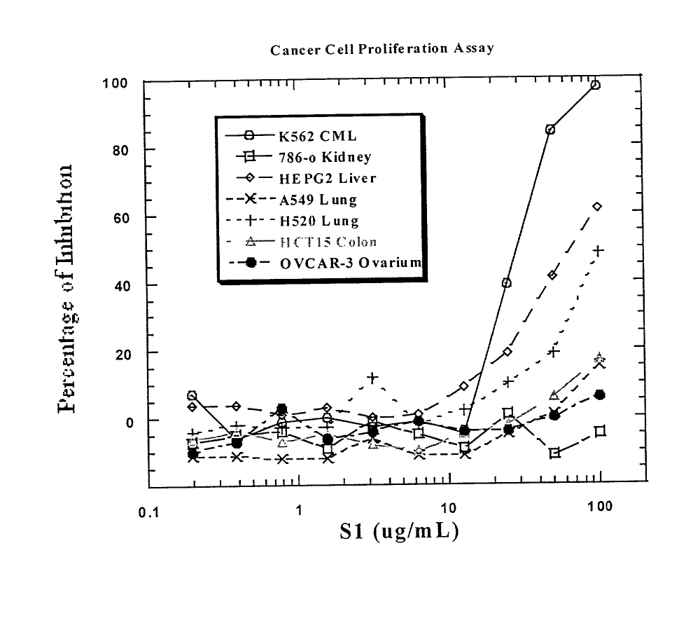

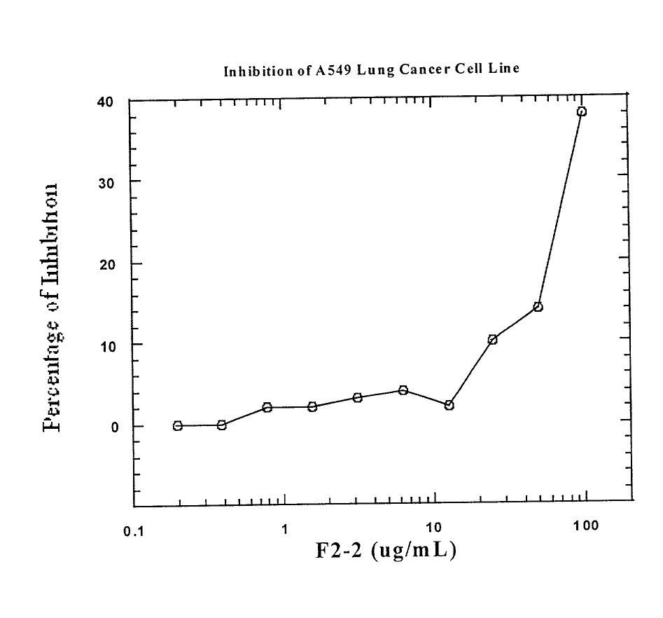

These novel compounds can be used for treatment of cancer. Particularly, the steroidal glycosides can be used to treat chronic myelogenous leukemia, liver, and lung cancer since these compounds showed anti-proliferation activity against cell lines from these diseases (FIG. 14). The flavonoid glycoside mixture F2-2, and the individual compounds contained in it, can be used to treat lung cancer as shown in FIG. 15. Treatment in accordance with the present invention can be accomplished by oral or intravenous administration along with a pharmaceutically acceptable carrier such as rice syrup solids, maltodextrin, and hydroxypropylcellulose, or in a food, to a patient having cancer. Orally-administrable dosage forms of the invention may include, but are not limited to, capsules, tablets, powders and liquids. The amount of active ingredient to be administered may vary depending upon the type of cancer and the body weight of the individual being treated. Dosages of the active ingredient may be in the range of 1 mg/kg per day to 30 mg/kg/day. These dosages should be continued until no detection of cancer is determined by standard means.

Another aspect of the present invention is a method as disclosed which utilizes oral or intravenous administration. However, those in the art will recognize that many avenues of administration are possible. For instance, administration of drug may be via capsule, tablet, solution, sachet, suspension, intravenously, orally, intramuscularly, including implantation into the tumor itself, topically or parenterally.

The present invention is believed to be utilizable with other types of cancers other than those described herein as would be known to those skilled in the art. The application of the presented method of extraction, purification and separation can be easily adopted to a preparation of pre-purified and standardized mixtures of phytochemicals or the individual compounds in a pure form for additional structural elucidation and/or conformation (e.g., by means of NMR spectroscopy or X-ray analysis) as well as for their screening for biological activity.

The presented invention does not require any additional steps, such as chemical derivation. However, it can be combined with either an analysis of partly and/or fully hydrolyzed material, as well as consecutive derivation of glycosides, and subjecting these samples to further HPLC/MSD/DAD or other methods of analysis. Applications of appropriate standards allow for an easy, sensitive and highly reliable method of quantitative analysis, and therefore, can be widely utilized for standardization of nutraceuticals.

Thus the presented procedure represents a much more industrially advantageous method for the execution of these analyses, particularly in the field of research, standardization and quality control of herbal and marine matter, or any of their nutraceutical supplement formulations.

### EXAMPLE 1

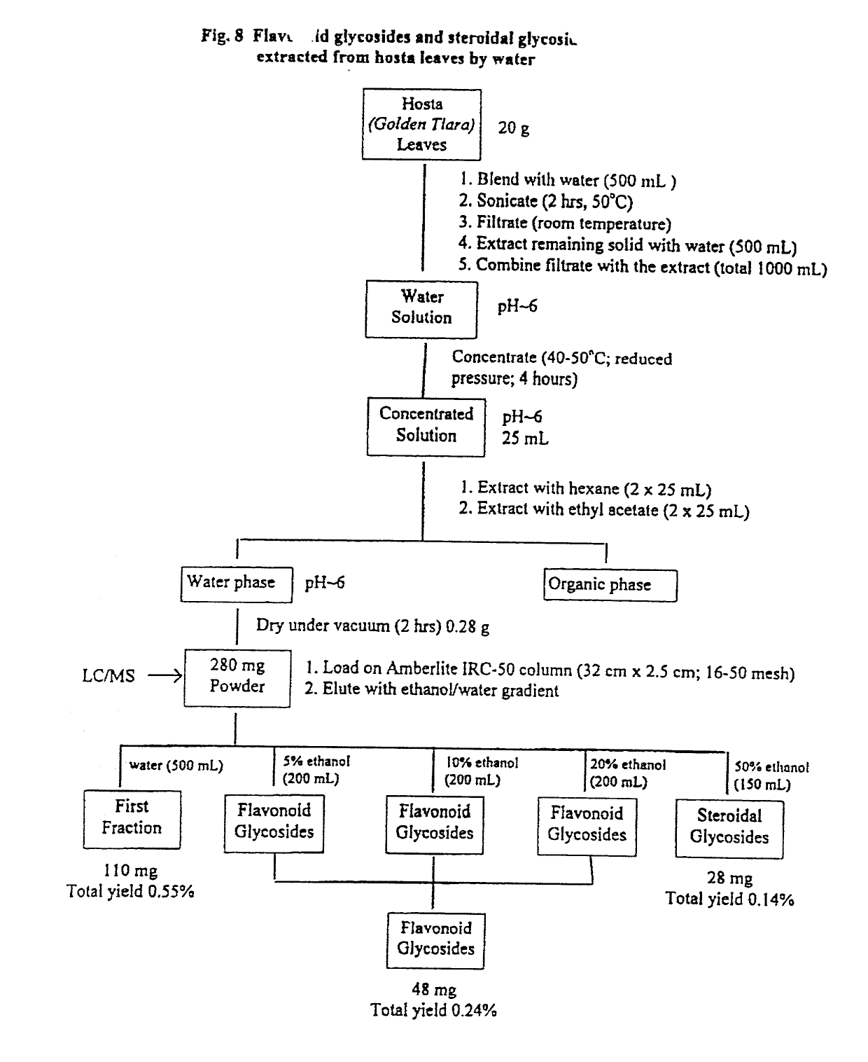

The procedure for extraction, pre-purification and analysis of Hosta leaves is depicted in FIG. 8. The fresh Hosta leaves (Golden Tiara) were hand picked in October. Fresh leaves (20 g) were chopped into small pieces and then ground in a blender with water (500 mL), followed by sonication for 2 hours at 50° C. The extract was filtrated to separate and remove fibrous material. The extraction of the separated solid material was then repeated with another portion of water (500 mL). Both extracts were combined and concentrated to ca. 25 mL under reduced pressure on a Rotovapor. The concentrated aqueous solution was extracted twice with hexane (25 mL) followed by two-time extraction with ethyl acetate (25 mL). The aqueous phase was evaporated under vacuum to dryness to afford 0.28 g (1.4% yield) of the raw extract of flavonoid and steroidal glycosides in the form of powder. This powder (0.28 g) was dissolved in 3.0 mL of a mixture of water and ethanol (1:1) and subjected to HPLC/MS analysis.

The solution of this raw extract was separated by a column chromatography on Amberlite IRC-50 resin (16-50 mesh, Sigma Company) with gradient elution of increasingly higher content of ethanol in water. The chromatography was monitored by HPLC/MSD/DAD. The first fraction eluted with water (500 mL) contained mainly some polar interfering compounds, such as sugars; the evaporation under vacuum to dryness yielded a solid powder (110 mg). The second fraction was eluted with 5% ethanol-water solution (200 mL). The third fraction was eluted with 10% ethanol-water solution (200 mL) and the fourth fraction eluted with 20% ethanol-water solution (200 mL). The second, third and fourth fractions were combined and evaporated under vacuum to dryness to afford 48 mg (0.24% yield) of a crude mixture of flavonoid glycosides. The fifth fraction was eluted with 50% ethanol-water solution (200 mL). This fraction was evaporated separately under vacuum to dryness to afford 28 mg (0.14% yield) of a crude mixture of steroidal glycosides.

The HPLC/MSD/DAD analysis was performed with a system that consisted of an HPLC 1100 series LC/MSD (Hewlett-Packard) instrument, autoinjector, quaternary pump with on-line vacuum degassing unit, thermostated column compartment and diode array detector. At the same time, a mass detector was used. The API-EI mode was chosen. The negative ion mode provided better sensitivity and the interpretation of the spectra was found to be easier. So the analysis results were obtained in negative mode at fragmentation potential of 100 cV. A standard Zorbax C8 column (150 mm long×2.1 mm I.D.) with 5 μm particle size was used in these examples.

Operation conditions for the analysis were as follows:

Temperature 30° C.

Mobile phase consisted of an ACN/water mixture gradient:

0-6 minutes ACN 15%,

6-18 minutes ACN from 15% to 90%

18-20 minutes ACN from 90% to 15%

The mobile phase flow rate was 0.4 mL/min.

Wavelength of UV detector was recorded on 280 nm.

The mass ion scan was from 100 to 1800.

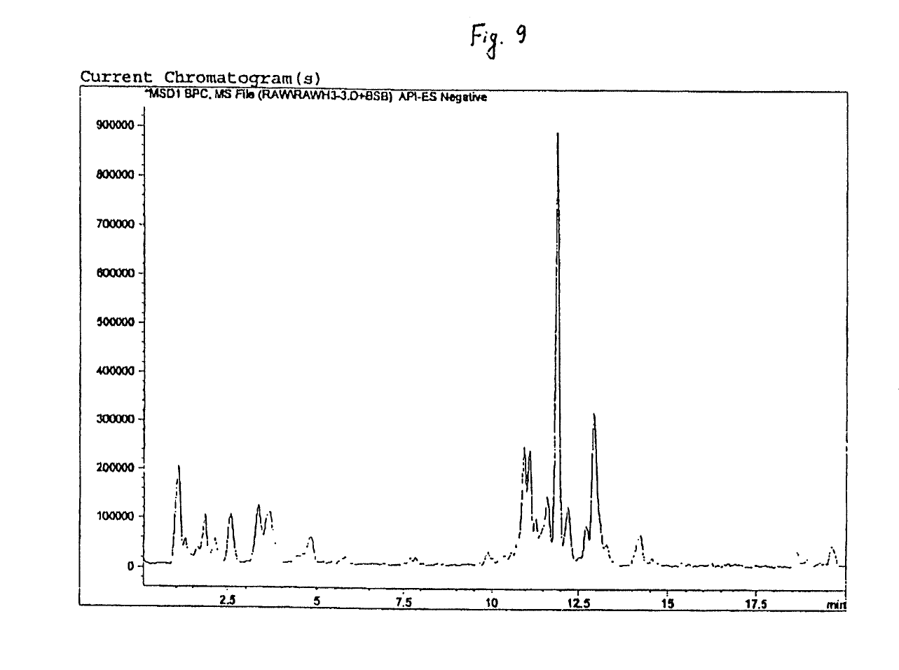

The recorded LC/MS chromatogram of the raw extract is presented in FIG. 9.

### EXAMPLE 2

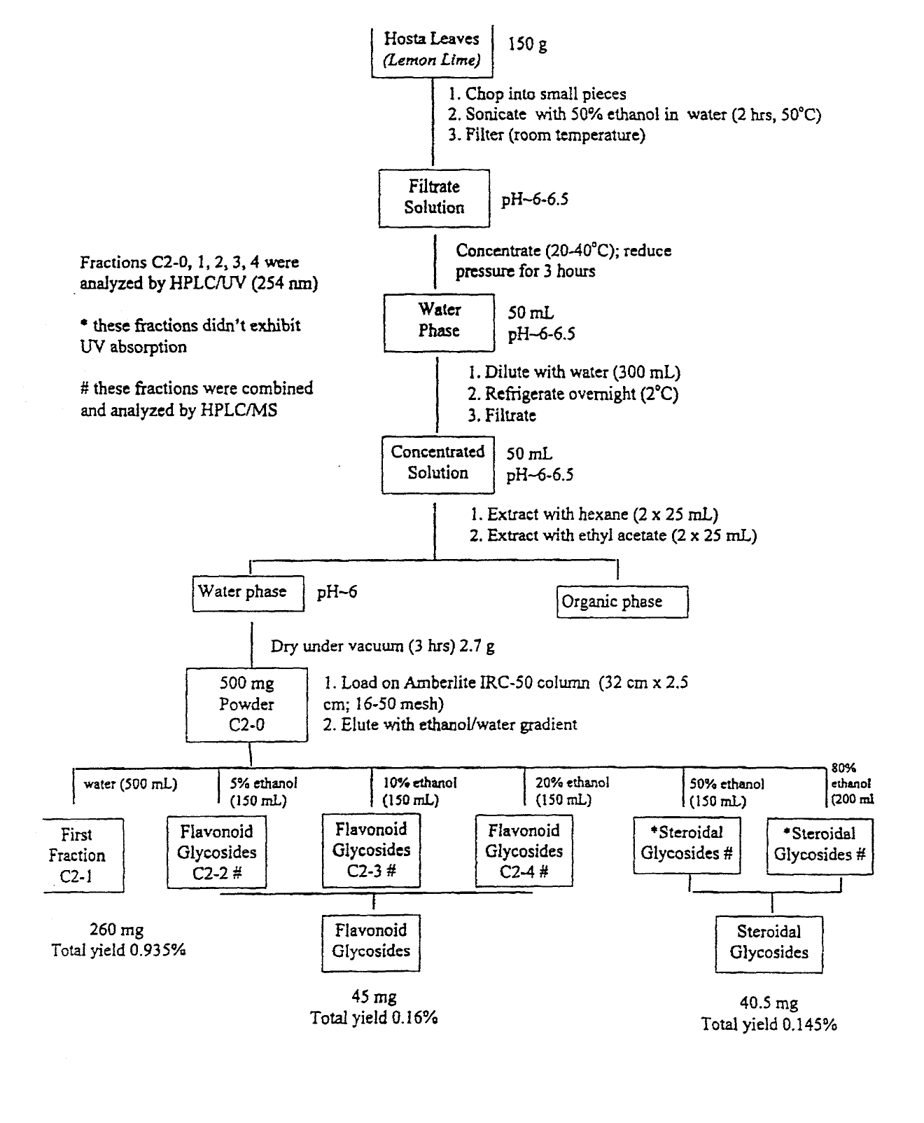

The procedure for extraction, pre-purification and analysis of Hosta leaves is depicted in FIG. 10. The fresh Hosta leaves (Lemon Lime) were hand picked in September. Fresh leaves (150 g) were chopped into small pieces and then ground in a blender with 50% aqueous ethanol solution (500 mL), followed by sonication for 2 hours at 50° C. The extract was filtrated to separate and remove fibrous material. The extract was concentrated to ca. 50 mL under reduced pressure on a Rotovapor. The concentrated aqueous solution was diluted to 300 mL with water, refrigerated overnight and the formed precipitate of undesired components such as alkylphenols and fat was filtered out. The resulted filtrate was extracted twice with hexane (25 mL) followed by two time extraction with ethyl acetate (25 mL). The aqueous phase was evaporated under vacuum to dryness to afford 2.7 g (1.8% yield) of the raw extract of flavonoid and steroidal glycosides in the form of powder. This powder (0.5 g) was dissolved in 5.0 mL of a mixture of water and ethanol (1:1) and subjected to chromatographic separation. The separation was performed by a column chromatography on Amberlite IRC-50 resin (16-50 mesh, Sigma Company) with gradient elution of increasingly higher content of ethanol in water. The chromatography was monitored by HPLC/UV and the analysis of the final combined fractions by HPLC/MSD/DAD. The first fraction eluted with water (500 mL) contained mainly some polar interfering compounds, such as sugars; the evaporation under vacuum to dryness yielded a solid powder (260 mg). The second fraction was eluted with 5% ethanol-water solution (150 mL). The third fraction was eluted with 10% ethanol-water solution (150 mL) and the fourth fraction eluted with 20% ethanol-water solution (150 mL). The second, third and fourth fractions were combined and evaporated under vacuum to dryness to afford 45 mg (0.16% yield) of a crude mixture of flavonoid glycosides. The fifth fraction was eluted with 50% ethanol-water solution (150 mL) and the sixth one with 80% ethanol-water solution (200 mL). Fractions five and six were combined and evaporated separately under vacuum to dryness to afford 40.5 mg (0.145% yield) of a crude mixture of steroidal glycosides.

The HPLC/MSD/DAD analysis was performed with a system that consisted of an HPLC 1100 series LC/MSD (Hewlett-Packard) instrument, autoinjector, quaternary pump with on-line vacuum degassing unit, thermostated column compartment and diode array detector. At the same time, a mass detector was used. The API-EI mode was chosen. The negative ion mode provided better sensitivity and the interpretation of the spectra was found to be easier. So the analysis results were obtained in negative mode at fragmentation potential of 400 eV. A standard Zorbax C8 column (150 mm long×2.1 mm I.D.) with 5 μm particle size was used in these examples. Operation conditions for the analysis were as follows: Temperature 30° C. Mobile phase consisted of an ACN/water mixture gradient:

0-15 minutes ACN 15%

15-20 minutes ACN from 15% to 90%

20-25 minutes ACN 90%

25-30 minutes ACN from 90% to 15%

The mobile phase flow rate was 0.4 mL/min.

Wavelength of UV detector was recorded on 280 nm.

The mass ion scan was from 100 to 1800.

The recorded LC/MS chromatogram of the pre-purified extract is presented in FIG.

### EXAMPLE 3

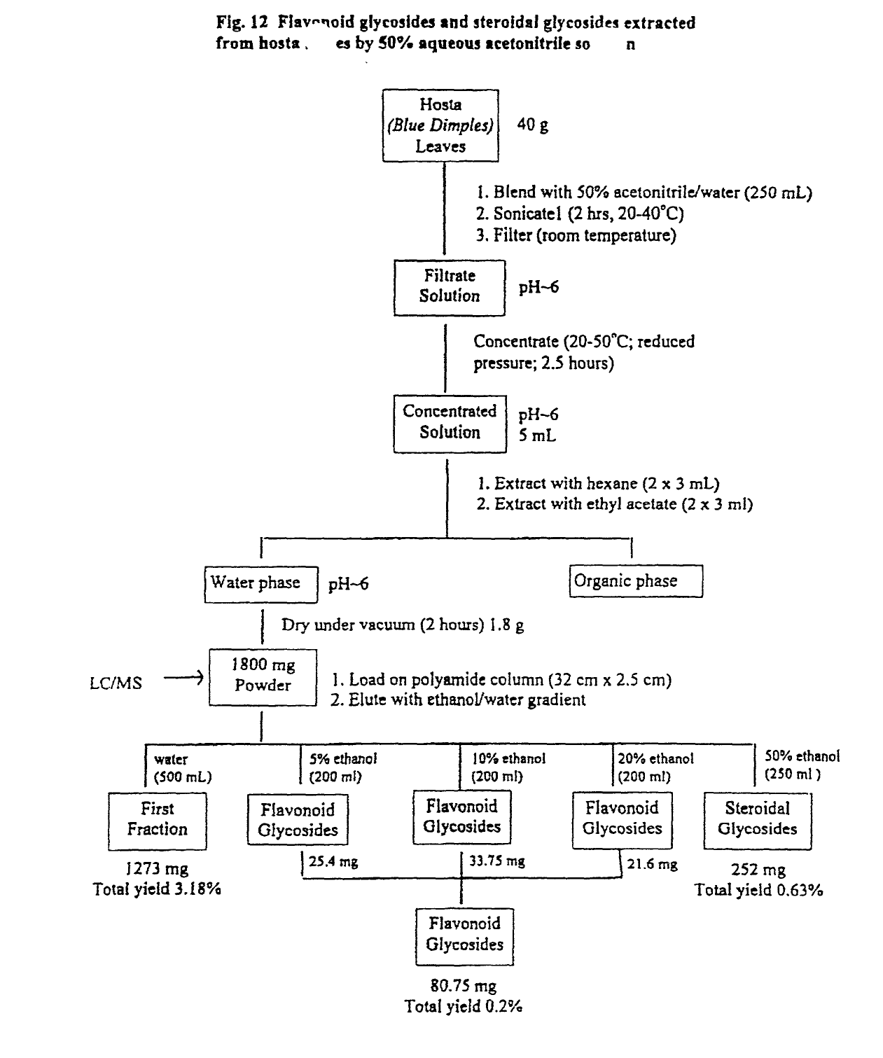

The procedure for extraction, pre-purification and analysis of Hosta leaves is depicted in FIG. 12. The fresh Hosta leaves (Blue Dimples) were hand picked in September. Fresh leaves (40 g) were chopped into small pieces and then ground in a blender with 50% acetonitrile solution in water (250 mL), followed by sonication for 2 hours at 20-40° C. The extract was filtrated to separate and remove fibrous material. The extract was concentrated to ca. 5 mL under reduced pressure on a Rotovapor. The concentrated solution was extracted twice with hexane (3 mL) followed by two-time extraction with ethyl acetate (3 mL). The aqueous phase was evaporated under vacuum to dryness to afford 1.8 g (4.5% yield) of the raw extract of flavonoid and steroidal glycosides in the form of powder. This powder (1.8 g) was dissolved in 10.0 mL of a mixture of water and acetonitrile (1:1) and subjected to HPLC/MS analysis.

The solution of this raw extract was separated by a column chromatography on polyamide resin (25 g, 80 mesh) with gradient elution of increasingly higher content of ethanol in water. The chromatography was monitored by HPLC/MSD/DAD. The first fraction eluted with water (500 mL) contained mainly some polar interfering compounds, such as sugars; the evaporation under vacuum to dryness yielded a solid powder (1273 mg). The second fraction was eluted with 5% ethanol-water solution (200 mL). The third fraction was eluted with 10% ethanol-water solution (200 mL) and the fourth fraction eluted with 20% ethanol-water solution (200 mL). The second, third and fourth fractions were combined and evaporated under vacuum to dryness to afford 80.75 mg (0.2% yield) of a crude mixture of flavonoid glycosides. The fifth fraction was eluted with 50% ethanol-water solution (250 mL). This fraction was evaporated separately under vacuum to dryness to afford 252 mg (0.63% yield) of a crude mixture of steroidal glycosides.

The HPLC/MSD/DAD analysis was performed with a system that consisted of an HPLC 1100 series LC/MSD (Hewlett-Packard) instrument, autoinjector, quaternary pump with on-line vacuum degassing unit, thermostated column compartment and diode array detector. At the same time, a mass detector was used. The API-EI mode was chosen. The negative ion mode provided better sensitivity and the interpretation of the spectra was found to be easier. So the analysis results were obtained in negative mode at fragmentation potential of 100 eV. A standard Zorbax C8 column (150 mm long×2.1 mm I.D.) with 5 μm particle size was used in these examples.

Operation conditions for the analysis were as follows: Temperature 30° C.

Mobile phase consisted of an ACN/water mixture gradient:

0-6 minutes ACN 15%

6-18 minutes ACN from 15% to 90%

18-20 minutes ACN from 90% to 15%

The mobile phase flow rate was 0.4 ml/min.

Wavelength of UV detector was recorded on 280 nm.

The mass ion scan was from 100 to 1800.

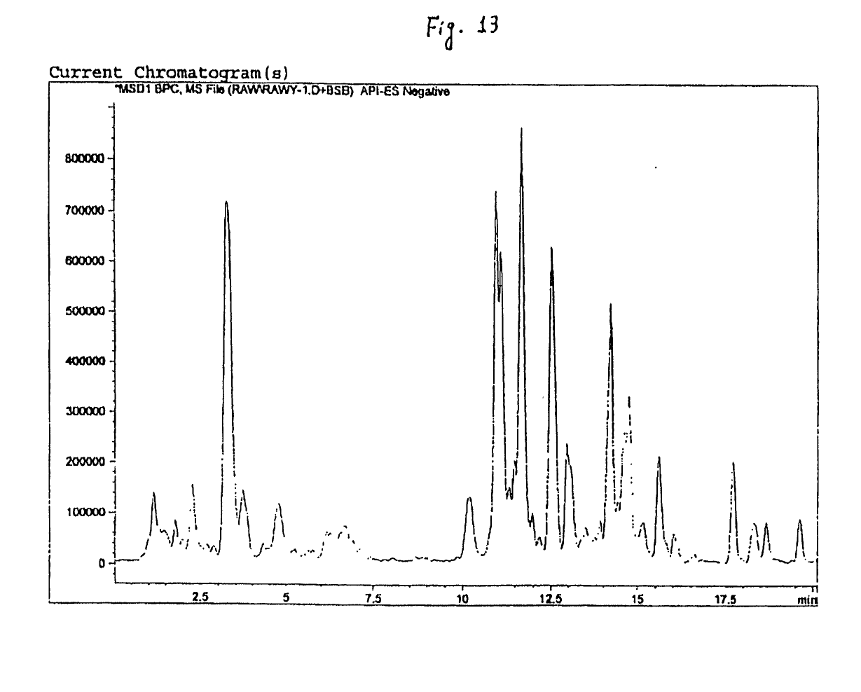

The recorded LC/MS chromatogram of the raw extract is presented in FIG. 13.

### EXAMPLE 4

These novel compounds were tested against seven cancer cell lines for anti-tumor activity. The assays were performed with extracts obtained one step prior to the final purification of individual compounds. The percentage of each component in the extracts was characterized. The anti-cancer activity of these compounds was examined over a wide concentration range between 100 ug/mL to 0.191 ng/mL. S1, the extract containing five steroidal glycosides wherein 53.8% is a compound of molecular weight of 1,406 Da, exhibits considerable anti-proliferation activity against three cell lines from CML, liver, and lung cancer (FIG. 14). One of the flavonoid glycoside mixture, F2-2, showed selective activity on inhibition of the lung cancer cell line A549 (FIG. 15). This mixture contains five flavonoid glycosides of molecular weight of 756, 918, 888, 726, and 902 Dalton respectively. The relative amount for each compound is 12.6, 39.9, 24.2, 14.5, 4.8, and 3.8%, respectively.

### References Cited

### U.S. Patent Documents

Ghai, G., et al, U.S. Pat. No. 5,955,269

Inada, S., et al., U.S. Pat. No. 4,968,787

Deninno, M. P., U.S. Pat. No. 5,698,526

Frazier, S. E., U.S. Pat. No. 4,238,483

### Foreign Patent Documents

Ochi, M., et al., Steroid Saponin from Hosta and Antimicrobial and Antitumor Agents Containing It. JP 10 114,791 [98 114,791] (C1. C07J71/00), May 6, 1998, Appl. 96/270,292, Oct. 11, 1996; 12 pp; CA 1129: 32293w

Ochi, M., et al., Novel Steroidal Saponin and Antimicrobial Agents and Antitumor Agents Containing It. JP 10 158,295 [98 158,295] (C1. C07J71), Jun. 16, 1998, Appl. 96/320, 142, Nov. 29, 1996; 12 pp; CA 129: 113511t

### Other Publications

(1) Li, Shih Zhen (Ming Dynasty), Ben Cao Gang Mu, Chinese Files Publisher, 1999 958-959

(2) Ghai, G., et al. U.S. Pat. No. 5,955,269.

(3) Catherine, A. Rice-Evans and Lester, Packer (eds), Flavonoids in Health and Disease, New York: Marcel Dekker 1998 61-110, 199-295

(4) Waller, R. and Dazuo Yamasaki (eds) Saponins in Traditional and Modern Medicine (Advances in Experimental Medicine and Biology, Vol. 404) New York and London: Plenum Press, 1996 1-14

(5) Maillard, M. P., et al. Journal of Chromatography A 1993, 647, 147-154.

(6) Games, D. E. Biomedical Mass Spectrometry 1981, 8, 454-462.

(7) Vaccaro, W. D., et al. Bioorganic & Medicinal Chemistry Letters 1998, 8, 313-318.

(8) Hostettmann, K and Marston, A., Saponins (Chemistry and Pharmacolog of Natural Product), Cambridge University, Cambridge, 1995 122-123, 128-139

(9) Maillard, M. P., et al. Journal of Chromatography A 1993, 647, 137-146.

(10) Lee, M., et al. Journal of Mass Spectrometry 1999, 34, 804-812.

(11) Deninno, M. P., U.S. Pat. No. 5,698,526

(12) Harborne, J. B., (ed) The Flavonoids: Advances in Research Since 1986, London: Chapman and Hall, 1994 586-618

(13) Harborne, J. B. F R S and Herbert, B., (eds) The Handbook of Natural Flavonoids, New York: John Wiley&Sons Inc. 1999 297-333

(14) Saija, A., et al, Free Radical Biology & Medicine 1995 19(4): 481-486

(15) Frazier, S. E., U.S. Pat. No. 4,238,483

(16) Dawidar, A. M., et al. Journal of Pharmaceutical Sciences 1974, 63, 140-142

(17) Lunte, S. M. Journal of Chromatography 1987, 384, 371-382.

(18) Agrawel, P. K., (ed) Carbon-13 NMR of Flavonoids, Elsevier, Amsterdam 1989

(19) Hasler, A., et al. Journal of Chromatography 1990, 508, 236-240.

(20) Inado, S., et al U.S. Pat. No. 4,968,787

(21) Wolfender, J. L., et al. Journal of Chromatography 1993, 647, 183-190.

(22) Pietta, P., et al. Journal of Chromatography A 1994, 661, 121-126.

(23) Wolfender, J. L., et al. Journal of Chromatography A 1995, 712, 155-168.

(24) Hakkinen, S., et al. Journal of Chromatography A 1998, 829, 91-100.

(25) Mauri, P. L., et al. Rapid Communications in Mass Spectrometry 1999, 13, 924-931

(26) Gelpi, E., Journal of ChromatographyA 1995, 703, 59-80.

(27) Yu, Chuan Long (eds), Zhong Yao Ci Hai ( Vol. 1), Chinese Medicine Technology Publisher, 1993 1347-1348

(28) Ochi, M., et al. Steroid Saponin from Hosta and Antimicrobial and Antitumor Agents Containing It 1998, JP 10 114,791 [198 114,791] (C1. C107J171/100), 116 May 1998, Appl. 1996/1270,1292, 1911 October 1996; 1912 pp; CA 1129: 32293w.

(29) Ochi, M., et al. Novel Steroidal Saponin and Antimicrobial Agents and Antitumor Agents Containing It 1998, JP 10 158,295 [198 158,295] (C1. C107J171), 116 June 1998, Appl. 1996/1320, 1142, 1929 November 1996; 1912 pp; CA 1129: 113511t.

(30) Budzianowski, J., et al. Phytochemistry 1990, 29, 3463-3467.

(31) Takeda, K., et al. Tetrahedron 1965, 21, 2089-2093.

(32) Takeda, K., et al. Journal of Chemical Society C 1967, 9, 876-882.

(33) Takeda, K., et al. Chemical and Pharmaceutical Bulletin 1968, 16, 275-279.

(34) Mimaki, Y., et al. Chemical and Pharmaceutical Bulletin 1995, 43, 1190-1196.

(35) Mimaki, Y., et al. Phytochemistry 1996, 42, 1065-1070.

(36) Mimaki, Y., et al. Phytochemistry 1997, 44, 305-310.

(37) Mimaki, Y., et al. Phytochemistry 1998, 48, 1361-1369.

What is claimed is:

1. A method of preventing or treating cancer by administering to a patient a therapeutically effective amount of a steroidal glycoside or mixtures thereof.

2. The method of claim 1 wherein the therapeutically effective amount of the steroidal glycoside is approximately 1 mg/kg of body weight per day to approximately 30 mg/kg body weight per day.

3. A method of preventing or treating cancer by administering to a patient a therapeutically effective amount of a flavonoid glycoside or mixtures thereof.

4. The method of claim 3 wherein the therapeutically effective amount of the flavonoid glycoside is approximately 1 mg/kg of body weight per day to approximately 30 mg/kg body weight per day.

5. The method of claim 1 wherein the steroidal tetraglycoside is of the molecular formula: C₅₁H₈₆O₂₃ (molecular weight of 1066) containing open spiro ring Tigogenin aglycone.

6. The method of claim 1 wherein the steroidal pentaglycoside is of the molecular formula: C₅₇H₉₄O₂₈ (molecular weight of 1226) containing Gitogenin aglycone.

7. The method of claim 1 wherein the steroidal pentaglycoside is of the molecular formula: C₅₇H₉₆O₂₉ (molecular weight of 1244) containing open spiro ring Gitogenin aglycone.

8. The method of claim 1 wherein the steroidal hexaglycoside is of the molecular formula: C₆₂H₁₀₄O₃₂ (molecular weight of 1360) containing open spiro ring Tigogenin aglycone.

9. The method of claim 1 wherein the steroidal hexaglycoside is of the molecular formula: C₆₂H₁₀₄O₃₃ (molecular weight of 1376) containing open spiro ring Gitogenin aglycone.

10. The method of claim 1 wherein the steroidal hexaglycoside is of the molecular formula: C₆₃H₁₀₆O₃₃ (molecular weight of 1390) containing open spiro ring Tigogenin aglycone.

11. The method of claim 1 wherein the steroidal hexaglycoside is of the molecular formula: C₆₃H₁₀₆O₃₄ (molecular weight of 1406) containing open spiro ring Gitogenin aglycone.

12. The method of claim 3 wherein the flavonoid tetraglycoside is of the molecular formula: C₃₉H₅₀O₂₄ (molecular weight of 902) containing Kaempferol aglycone.
